(19) United States
(12) Patent Application Publication
(10) Pub. No.: US 2019/0021633 A1
(43) Pub. Date: Jan. 24, 2019

(54) DETECTING RESPIRATORY RATES IN AUDIO USING AN ADAPTIVE LOW-PASS FILTER

(71) Applicants: Ling Wang, San Jose, CA (US); Karolina Cengija, Hillsboro, OR (US); Michael R. Rosen, San Jose, CA (US); Naghma Anwar, San Jose, CA (US); Thao-Vy Nguyen, San Francisco, CA (US)

(72) Inventors: Ling Wang, San Jose, CA (US); Karolina Cengija, Hillsboro, OR (US); Michael R. Rosen, San Jose, CA (US); Naghma Anwar, San Jose, CA (US); Thao-Vy Nguyen, San Francisco, CA (US)

(21) Appl. No.: 15/819,220

(22) Filed: Nov. 21, 2017

(51) Int. Cl. A61B 5/08; A61B 5/00

(52) U.S. Cl. CPC A61B 5/0816; A61B 5/0871; A61B 5/725

(57) ABSTRACT

An example apparatus for detecting respiratory rates includes an audio receiver to receive audio including respiratory sounds. The apparatus also includes an adaptive low-pass filter to process the audio using an adaptive bandwidth based on a mean breath rate to generate an envelope. The apparatus further includes a peak detector to detect a plurality of peaks from the envelope. The apparatus includes a respiratory rate calculator to calculate a respiratory rate based on the detected plurality of peaks.

### BACKGROUND

Respiratory rate measuring instruments may be used to detect patterns of respiration. For example, such respiratory rate measuring instruments may use a flow-rate and pressure-relationship, a pressure and temperature change, infrared imaging techniques, or oxygen and carbon dioxide amount change analysis to detect changes in respiratory rate of a monitored individual.

### BRIEF DESCRIPTION OF THE DRAWINGS

FIG. 1 is a block diagram illustrating an example system for detecting respiratory rates in audio signals using an adaptive low-pass filter;

FIG. 2 is a detailed flow chart illustrating an example process for detecting respiratory rates in audio signals using an adaptive low-pass filter;

FIG. 3 is a diagram illustrating an example preprocessed audio signal with a corresponding envelope;

FIG. 4A is a diagram illustrating an example raw audio signal;

FIG. 4B is a diagram illustrating an example preprocessed audio signal;

FIG. 4C is a diagram illustrating an example envelope with a detected breath;

FIG. 5 is a diagram illustrating example set of detection results;

FIG. 6 is a flow chart illustrating a method for detecting respiratory rates in audio signals using an adaptive low-pass filter;

FIG. 7 is block diagram illustrating an example computing device that can detect respiratory rates in audio signals using an adaptive low-pass filter; and

FIG. 8 is a block diagram showing computer readable media that store code for detecting respiratory rates in audio signals using an adaptive low-pass filter.

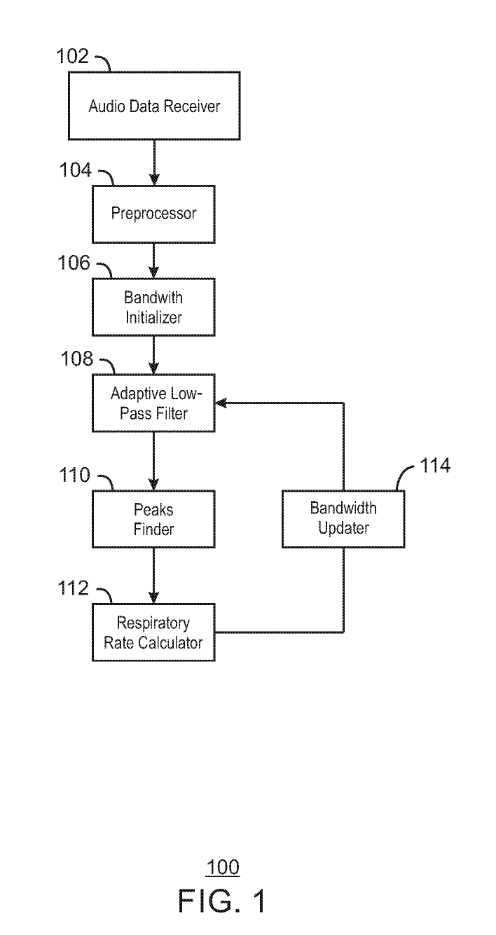

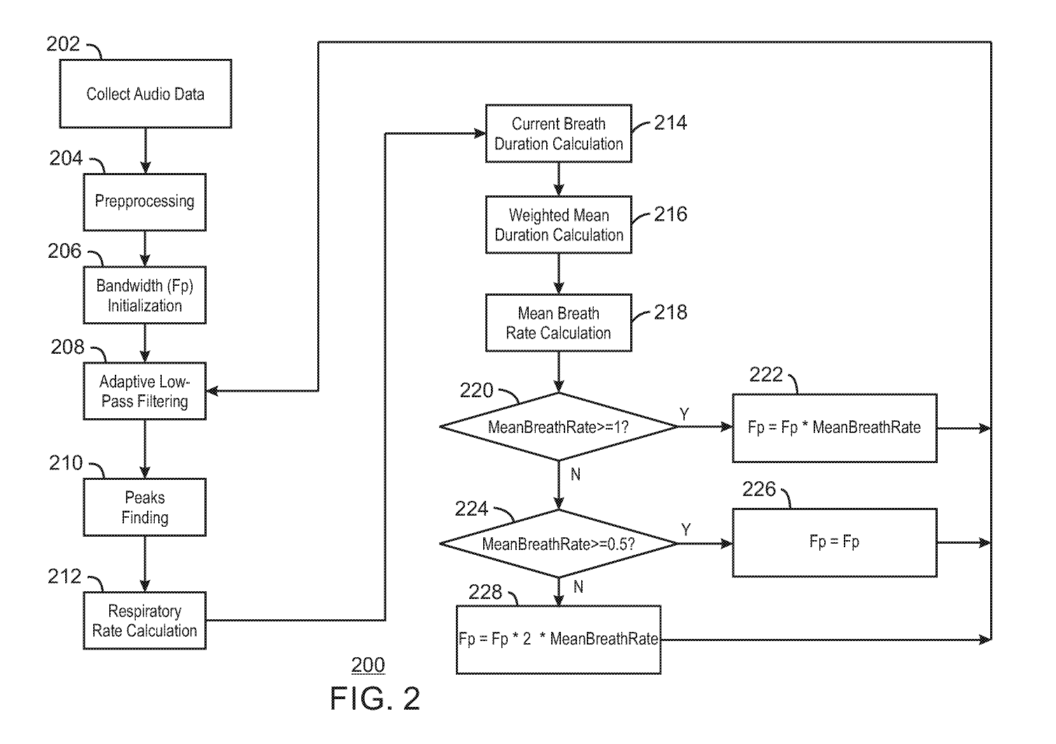

The same numbers are used throughout the disclosure and the figures to reference like components and features. Numbers in the 100 series refer to features originally found in FIG. 1; numbers in the 200 series refer to features originally found in FIG. 2; and so on.

### DESCRIPTION OF THE EMBODIMENTS

As discussed above, respiratory rate measuring instruments may use various techniques to detect patterns of respiration. For example, some instruments may use a flow-rate and pressure-relationship, a pressure and temperature change, infrared imaging techniques, oxygen and carbon dioxide amount change. However, such respiratory rate measuring instruments may be designed for use in a hospital. For example, the respiratory rate measuring instruments may be usually large in size, high in price, complex, or otherwise inconvenient to use at home. Moreover, such respiratory rate measuring instruments may also not be portable.

In some examples, audio of respiration may be captured and used to detect respiratory rates. For example, a low-pass filter may be used to generate a signal envelope in an audio signal containing respiratory sounds. As used herein, an envelope refers to smooth curve outlining extremes in an audio sample. However, setting the low-pass filter to a bandwidth that is too low or too high may result in inaccurate detections or failed detections. For example, if the bandwidth is too low, then the envelope may not be able to distinguish fast breathing and may cause potential detections to be missed. On the other hand, if the bandwidth is too high, there may be more spikes in the audio signal of slower breathing that may cause false detections. As used herein, a spike refers to a sudden steep increase followed by a sudden steep drop in amplitude of an audio signal. Audio of respiration may refer to the audio signals created as a result of respiration.

The present disclosure relates generally to techniques for detecting respiratory rates in audio. Specifically, the techniques described herein include an apparatus, method, and system for detecting respiratory rates in audio signals using an adaptive low-pass filter. For example, the adaptive low-pass filter may have an adaptive bandwidth that may be adaptive to a detected mean breath rate. An example apparatus includes an audio receiver to receive audio including respiratory sounds. The apparatus includes an adaptive low-pass filter to process the audio using a bandwidth based on a mean breath rate to generate an envelope. The apparatus further includes a peak detector to detect a plurality of peaks from the envelope. The apparatus also includes a respiratory rate calculator to calculate a respiratory rate based on the detected plurality of peaks. In some examples, the apparatus further includes an audio preprocessor to preprocess the audio to generate preprocessed audio, the preprocessed audio to be processed to generate the envelope. The apparatus can also further include a filter bandwidth updater to update a bandwidth of the adaptive low-pass filter based on an updated mean breath rate.

The techniques described herein thus enable a dynamic bandwidth that significantly improves the accuracy of respiratory detection in audio signals. For example, adapting the bandwidth of the low-pass filter to the audio signal may reduce false detections while preventing missed detections. Moreover, the detected patterns of respiration using the techniques described herein may be used to enable health and work benefits. In some examples, respiratory rates may be increased by stress or anxiety. For example, stress may cause changes in one's mind and body and this will be reflected in respiratory rate. By knowing more about personal breathing patterns, a user can discover stress factors, factors that engage the user, when normal stress of the user turns into anxiety or worry, and apply breathing management to help control stress levels. The techniques may thus provide a mobile solution of monitoring respiratory rates and thus stress levels in users.

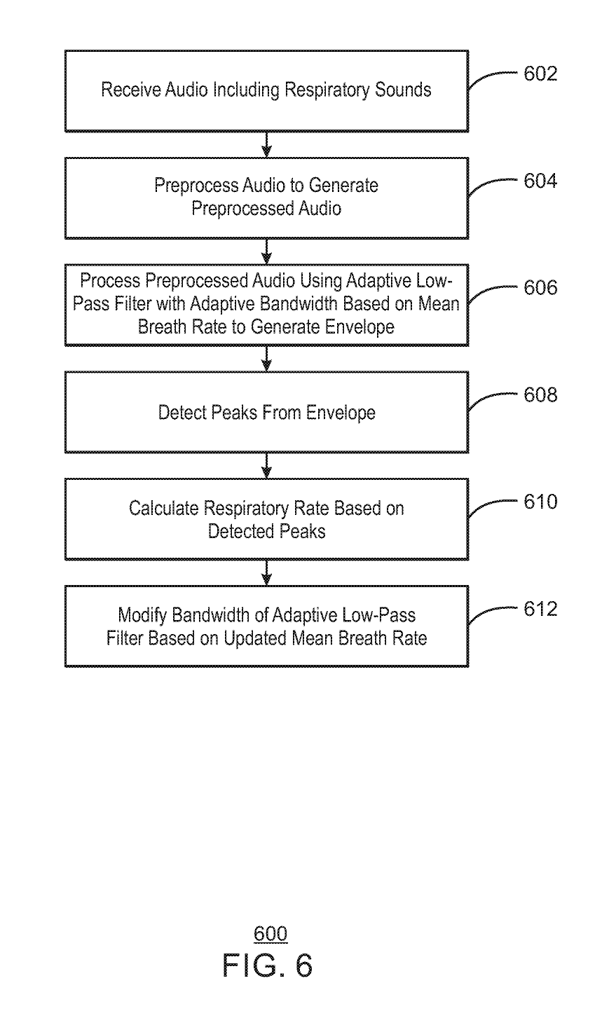

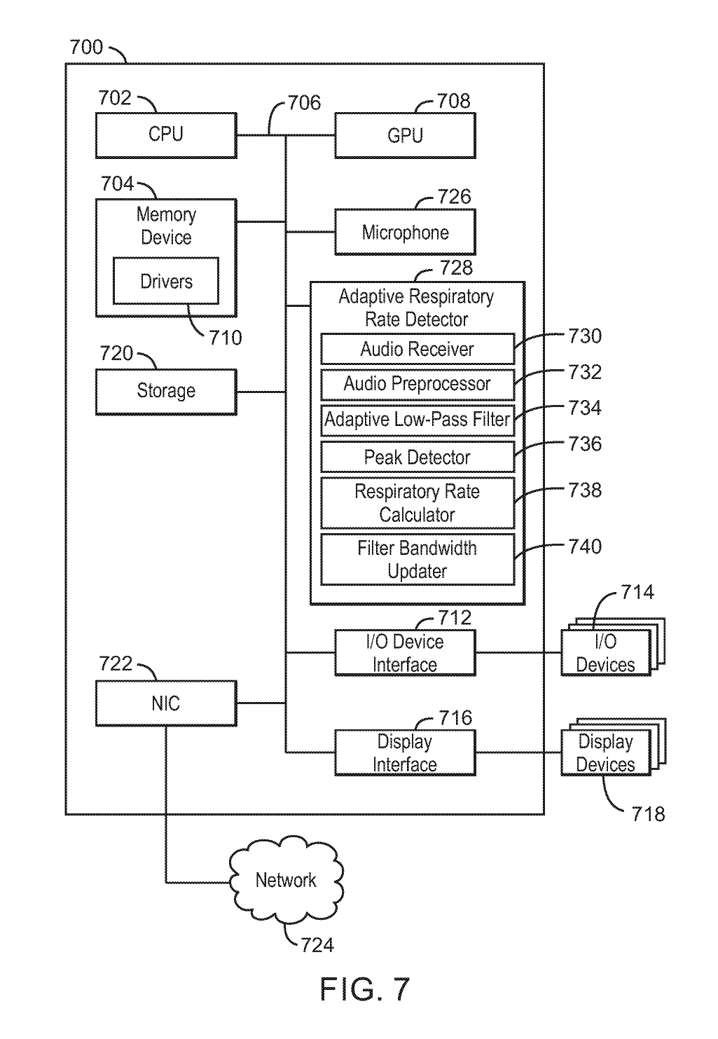

FIG. 1 is a block diagram illustrating an example system for detecting respiratory rates in audio signals using an adaptive low-pass filter. The example system is referred to generally by the reference number 100 and can be implemented in the computing device 700 below in FIG. 7 using the method 600 of FIG. 6 below.

The example system 100 includes an audio data receiver 102. For example, the audio data receiver may receive audio including respiratory sounds. The system 100 also includes a preprocessor 104 that is communicatively coupled to audio data receiver 102. For example, the preprocessor 104 may preprocess audio to remove noise. The preprocessor 104 is communicatively coupled to a bandwidth initializer 106. For example, the bandwidth initializer 106 may set an initial value for a bandwidth of an adaptive low-pass filter 108. The adaptive low-pass filter 108 is communicatively coupled to the bandwidth initializer 106. In some examples, the adaptive low-pass filter 108 can be used to generate envelopes from a received audio based on the bandwidth value. The system 100 also includes a peaks finder 110 that is communicatively coupled to the adaptive low-pass filter 108. For example, the peaks finder 110 may be used to detect peaks in envelopes generated by the adaptive low-pass filter 108. The system 100 further includes a respiratory rate calculator 112 that is communicatively coupled to the peaks finder 110 that can calculate a respiratory rate based on the detected peaks. The system also further includes a bandwidth updater 114 that is communicatively coupled to both the respiratory rate calculator 112 and the adaptive low-pass filter 108.

As shown in FIG. 1, the audio data receiver 102 may receive audio including respiratory sounds. For example, the respiratory sounds may be breathing captured by a microphone in a device (not shown). For example, the device may be a mobile device, such as a cellphone or a tablet. In some examples, the device can be a pair of smart glasses with an embedded microphone.

The preprocessor 104 can process the audio to make the audio more suitable for envelope generation and peak finding. For example, the preprocessor 104 can remove noise from the audio or reduce a DC offset of the audio. As used herein, a DC offset refers to a mean value of the waveform of the audio. For example, if the mean amplitude of an audio waveform is zero, then there is no DC offset. In some examples, the preprocessor 104 can also take an absolute value of the audio to generate a preprocessed audio, as described in greater detail below.

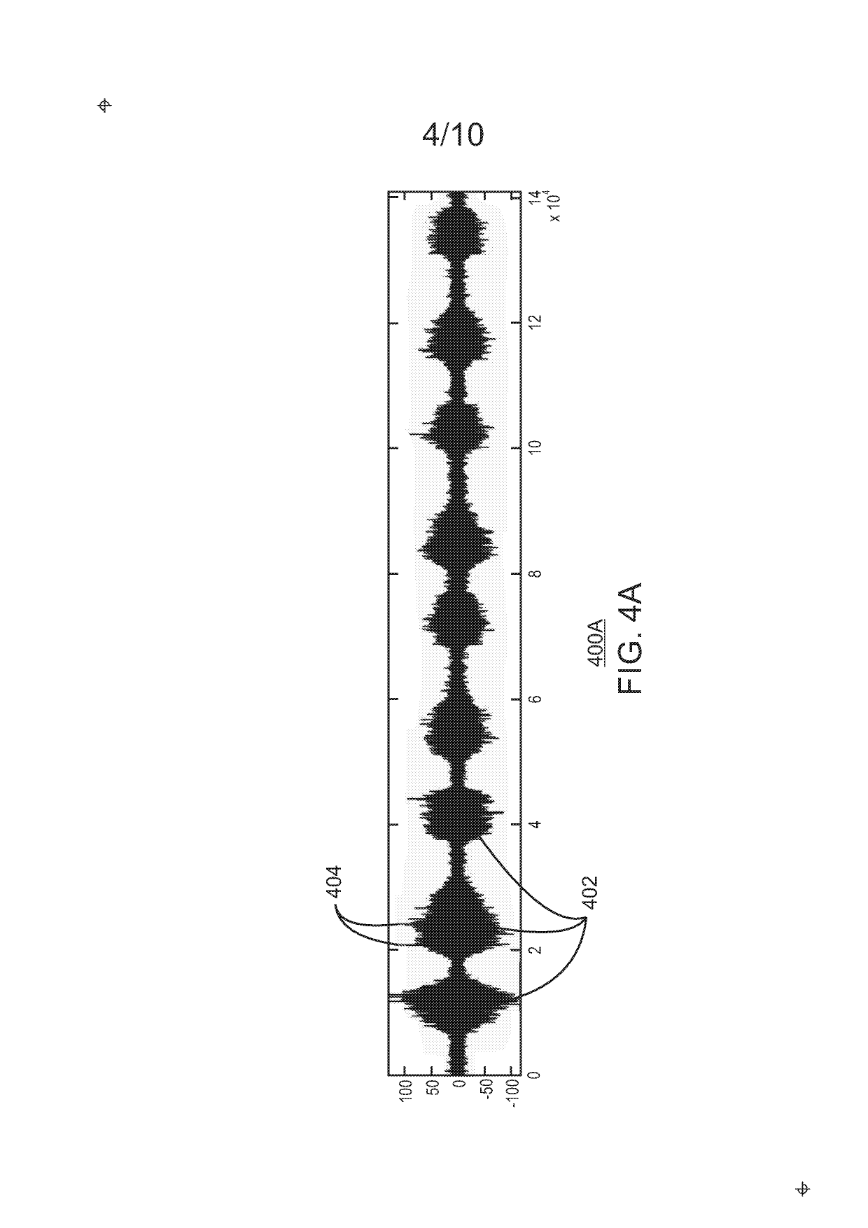

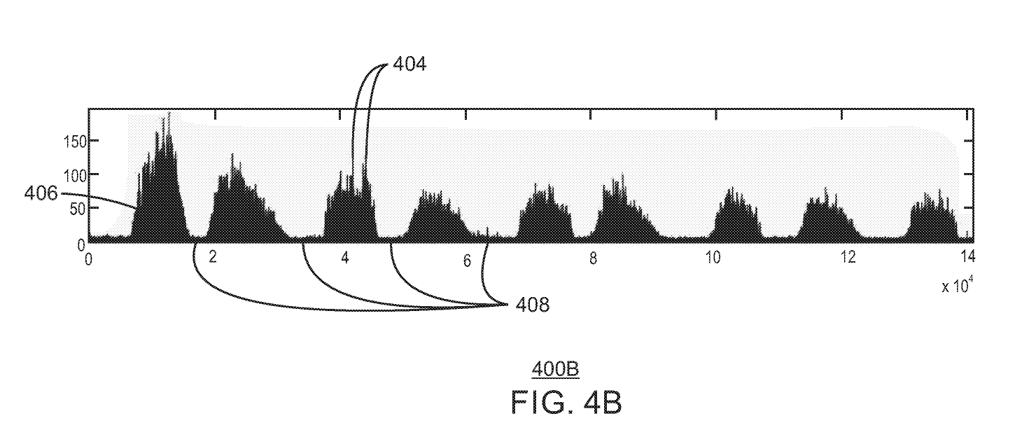

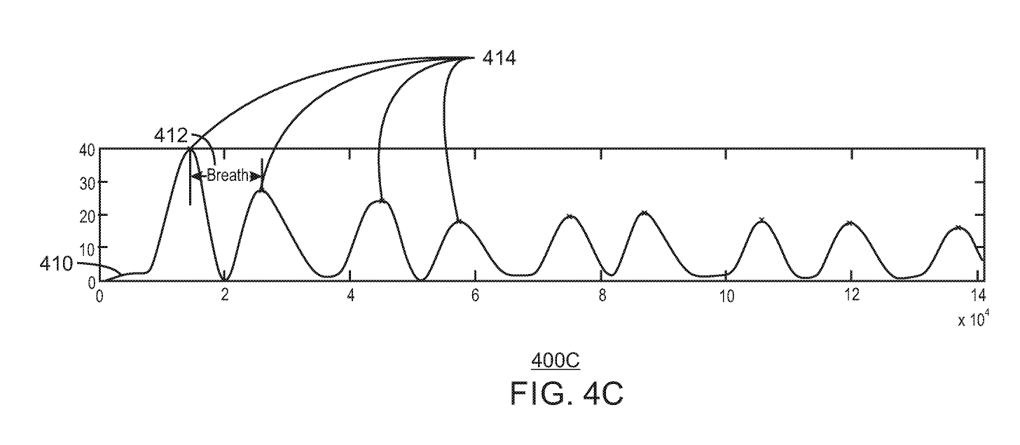

The bandwidth initializer 106 can set an initial bandwidth for the adaptive low-pass filter 108. For example, given nine breaths and a breath duration of about 14,000 milliseconds, the average duration of a breath in FIG. 4C may be about 14000/9=1555 milliseconds. A corresponding average breathing rate may be 0.65 Hz. In some examples, the initial bandwidth set by the bandwidth initializer may be based on an average breathing rate. For example, an initial bandwidth of the adaptive low-pass filter 108 may be approximately Fp=1.5 Hertz (Hz). In some examples, the initial value set by the bandwidth initializer 106 may be based on an activity of the user. For example, a set of preset initial values may be selected from based on the detected activity.

The adaptive low-pass filter 108 may then generate an envelope for the received audio. For example, the envelope may be generated based on the initial bandwidth value.

The peaks finder 110 can then detect peaks in the envelope. For example, one peak may be detected for each inhalation and exhalation sound in the audio.

The respiratory rate calculator 112 can then detect a respiratory rate based on the detected peaks. For example, the respiratory rate calculator 112 can detect a breath for every two detected peaks. In some examples, a duration of a breath may be the time between two consecutive peaks. As used herein, a breath refers to an inhalation or an exhalation, rather than a full breathing cycle that includes both an inhalation and an exhalation.

The bandwidth updater 114 can update the bandwidth of the adaptive low-pass filter 108. For example, the bandwidth updater 114 can update the bandwidth of the adaptive low-pass filter 108 in real-time. In some examples, if the breathing rate of a user is much higher or lower than 0.65 Hz, or the breathing rate of a user moves up or down, then the bandwidth of the adaptive low-pass filter 108 may be updated.

The diagram of FIG. 1 is not intended to indicate that the example system 100 is to include all of the components shown in FIG. 1. Rather, the example system 100 can be implemented using fewer or additional components not illustrated in FIG. 1 (e.g., additional preprocessors, filters, etc.).

FIG. 2 is a detailed flow chart illustrating an example process for detecting respiratory rates in audio signals using an adaptive low-pass filter. The example process is generally referred to by the reference number 200 and can be implemented in the system 100 above in FIG. 1 or using the processor 702 of the computing device 700 of FIG. 7 below.

At block 202, a processor can collect audio data. For example, an audio sample may be captured by a microphone. The audio sample may include respiratory sounds corresponding to the breathing of a user.

At block 204, the processor performs preprocessing on the audio data. For example, the preprocessing may be applied to the audio data in order to remove noise. In some examples, a difference may be calculated between two data in response to detecting that there is a DC offset in an audio. In some examples, the absolute value of the audio signal may be calculated. In some examples, a band stop filter may be applied to remove a certain frequency interference. For example, AC interference may be at 50 Hz or 60 Hz.

At block 206, the processor performs a bandwidth initialization. For example, an initial value for the bandwidth to be used by an adaptive low-pass filter may be set. In some examples, the initial value set for the bandwidth may be based on an average breath rate. For example, the average duration of a breath in FIG. 4C is about 14000/9=1555 ms, resulting in a breathing rate of about 0.65 Hz. Thus, in some examples, the bandwidth of the low pass filter may be set to an initial value of Fp=1.5 Hz in accordance with the Nyquist Theorem as discussed in greater detail below. In some examples, the initial value of the bandwidth of the adaptive low-pass filter may be set based on a detected activity of the user.

At block 208, the processor causes an adaptive low-pass filtering. For example, an adaptive low-pass filter can apply the low-pass filtering on audio or preprocessed audio to generate an envelope corresponding to the audio. In some examples, the envelope may be based on different bandwidth values depending on a mean breath rate, as described in greater detail below.

At block 210, the processor performs a peak finding algorithm. For example, one or more peaks may be detected in the envelope generated at block 208. In some examples, the one or more peaks can be detected using one or more adaptive thresholds. For example, the thresholds can be based on peak amplitude and distance between detected peaks.

At block 212, the processor performs a respiratory rate calculation. For example, the respiratory rate calculation may divide the total number of peaks over a period of time. For example, the respiratory rate may measure the total number of breaths (inhalations or exhalations) in a given period of time.

At block 214, the processor receives a current breath duration. For example, the current breath duration can have been calculated by the duration between the current and the previous peak.

At block 216, the processor performs a weighted mean duration calculation. For example, the current weighted mean duration can be calculated by the weighted mean of the current duration and a previously calculated weighted mean.

At block 218, the processor performs a mean breath rate calculation. For example, the mean breath rate can be calculated by 1 second divided by the weighted mean duration from block 216.

At decision diamond 220, the processor can determine whether mean breath rate is equal to or greater than the value one. If the mean breath rate is equal to or greater than the value one, then the process may continue at block 222. If the mean breath rate is not equal to or greater than the value one, then the process may continue at decision diamond 224.

At block 222, the processor increases the bandwidth in response to detecting that the mean breath rate has increased. For example, the processor can calculate the new bandwidth based on the equation:

F_{Pnew}=F_{Pold}*MeanBreathRate Eq. 1

where the MeanBreathRate is equal to or greater than one, F_{Pnew} is a newly calculated bandwidth for an adaptive low-pass filter, and F_{Pold} is a previously used bandwidth of the adaptive low-pass filter.

At decision diamond 224, the processor can determine whether mean breath rate is equal to or greater than the value 0.5. For example, if the mean breath rate is equal to or greater than the value 0.5, then the process may continue at block 226. If the mean breath rate is not equal to or greater than 0.5, then the process may continue at block 228.

At block 226, the processor sets the new bandwidth to the same value as the old bandwidth in response to detecting a breath rate between 0.5 and 1. In some examples, the processor may not update the bandwidth of the adaptive low-pass filter such that the adaptive low-pass filter continues to use the same bandwidth.

At block 228, processor decreases the bandwidth in response to detecting a mean breathing rate less than 0.5. For example, the processor can decrease the bandwidth based on the equation:

F_{Pnew}=F_{Pold}*2*MeanBreathRate Eq. 2

where the MeanBreathRate is less than 0.5.

Thus, as described above, when a user is breathing faster, the bandwidth may be increased such that the resulting envelope has more detail. On the other hand; when the user is breathing slower, the bandwidth may be reduced such that the envelope is not too spiky to avoid false detections of peaks. Using such an adaptive bandwidth for the adaptive low-pass filter, the accuracy of the detection of peaks and thus respiratory rate may be significantly improved.

This process flow diagram is not intended to indicate that the blocks of the example process 200 are to be executed in any particular order, or that all of the blocks are to be included in every case. Further, any number of additional blocks not shown may be included within the example process 200, depending on the details of the specific implementation. For example, although three ranges for the mean bandwidth are use above, including >1, between 0.5 and 1, and <0.5, in some examples, the number of cases and the numbers (0.5, 1, 2 . . . ) can be further fine-tuned. For example, the ranges can be divided into additional ranges, or F_{Pnew}=F_{Pold}*1.05*MeanBreathRate in some examples, and F_{Pnew}=F_{Pold}*1.12*MeanBreathRate in other examples.

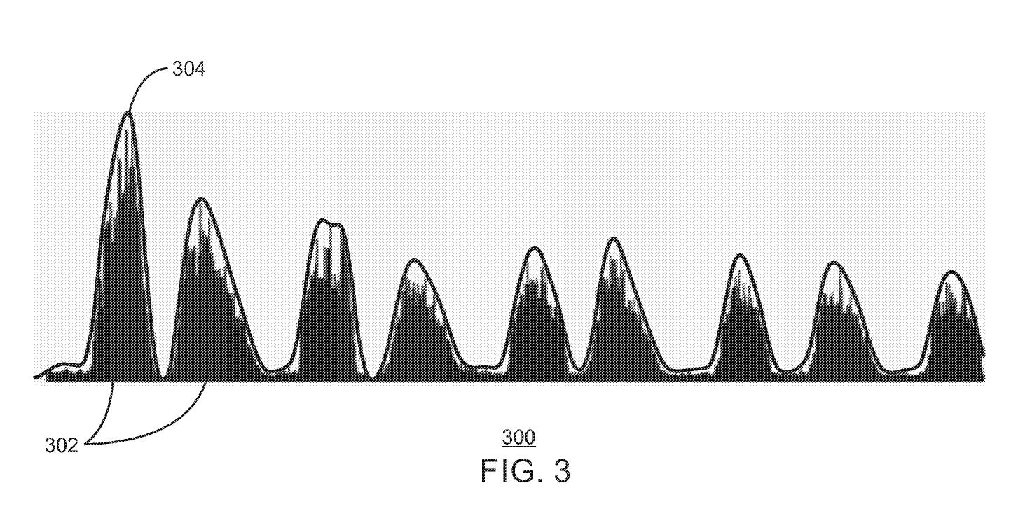

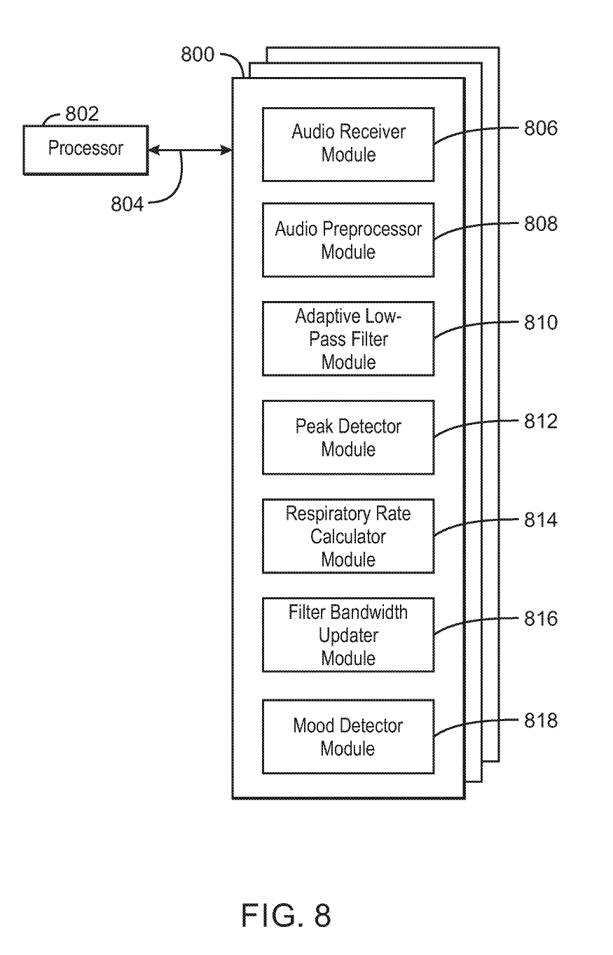

FIG. 3 is a diagram illustrating an example preprocessed audio signal with a corresponding envelope. The example preprocessed audio signal is generally referred to by the reference number 300 and can be generated by in the computing device 700 below. For example, the preprocessed audio signal 300 can be generated using the preprocessor 104 of the system 100 of FIG. 1, the audio preprocessor 732 of the computing device 700 of FIG. 7 below, or the audio preprocessor module 808 of the computer readable media 800 of FIG. 8 below.

FIG. 3 shows a set of preprocessed signals 302 that have been preprocessed as described above. For example, the preprocessed signals 302 may have been processed by a preprocessor to take an absolute value. In addition, the audio may have been processed to remove noise. As shown in FIG. 3, an envelope 304 may have been generated by an adaptive low-pass filter as described above. In some examples, peak finding may be performed on the envelope as described above and below. For example, since the envelope may have only one local maximum associated with each of the breaths in the preprocessed signals 302, peak finding may be performed more accurately on the generated envelope 304.

The diagram of FIG. 3 is not intended to indicate that the example preprocessed audio signal 300 is to include all of the components shown in FIG. 3. Rather, the example preprocessed audio signal 300 can be implemented using fewer or additional components not illustrated in FIG. 3 (e.g., additional preprocessing, preprocessed signals, etc.).

FIG. 4A is a diagram illustrating an example raw audio signal. The example raw audio signal is generally referred to by the reference number 400A and can be implemented in the computing device 700 below. For example, the raw audio signal 400A can be received at the audio data receiver 102 of the system 100 of FIG. 1, the audio receiver 730 of the computing device 700 of FIG. 7 below, or the audio receiver module 806 of the computer readable media 800 of FIG. 8 below.

FIG. 4A shows a set of sounds 402. As shown in FIG. 4A, the sounds 402 may include multiple spikes 404 indicating louder portions of the sounds 402. In some examples, the spikes 404 may be falsely detected as two separate respiratory sounds if a peak finding was performed directly on the raw audio signal 400A. Therefore, in some examples, preprocessing and envelope generation may be performed on the raw audio 400A, as described in FIGS. 4B and 4C below.

The diagram of FIG. 4A is not intended to indicate that the example raw audio signal 400A is to include all of the components shown in FIG. 4A. Rather, the example raw audio signal 400A can be implemented using fewer or additional components not illustrated in FIG. 4A (e.g., additional waveforms, audio signals, etc.).

FIG. 4B is a diagram illustrating an example preprocessed audio signal. The example preprocessed audio signal is generally referred to by the reference number 400B and can be implemented in the computing device 700 below. For example, the preprocessed audio signal 400B can be generated by the preprocessor 104 of the system 100 of FIG. 1, the audio preprocessor 732 of the computing device 700 of FIG. 7 below, or the audio preprocessor module 808 of the computer readable media 800 of FIG. 8 below.

The preprocessed audio signal 400B of FIG. 4B includes a set of preprocessed sounds 406 corresponding to sounds 402. The preprocessed sounds 406 may be the absolute value of the sounds 402 of the raw audio signal 400A. In addition, the preprocessed audio signal 400B includes gaps 408 with reduced noise. For example, a difference may have been taken between two audio signals to remove noise from the preprocessed audio signal 400B. As shown in FIG. 4B, each of the sounds 404 may still include multiple peaks. Therefore, in some examples, an envelope may be generated as described in FIG. 4C below.

The diagram of FIG. 4B is not intended to indicate that the example preprocessed audio signal 400B is to include all of the components shown in FIG. 4B. Rather, the example preprocessed audio signal 400B can be implemented using fewer or additional components not illustrated in FIG. 4B (e.g., additional processing, sounds, etc.).

FIG. 4C is a diagram illustrating an example envelope with a detected breath. The example detected respiratory rate is generally referred to by the reference number 400C and can be detected using the system 100 above or the computing device 700 below. For example, the detected respiratory rate 400C can be detected by the adaptive low-pass filter 108 of the system 100 of FIG. 1, the adaptive low-pass filter 734 of the computing device 700 of FIG. 7 below, or the adaptive low-pass filter module 810 of the computer readable media 800 of FIG. 8 below.

FIG. 4C includes an envelope 410. For example, the envelope 410 may have been generated by an adaptive low-pass filter. In some examples, the adaptive low-pass filter may have a bandwidth that is based on a mean breath rate. For example, the mean breath rate can be a weighted mean breath rate.

As shown in FIG. 4C, a breath 412 may be detected between two of a number of nine total detected peaks 414. For example, each of the detected peaks 414 may be associated with one of the sounds 408 of example 400B above. Thus, each of the waveforms may now be associated with one detected peak 414. In some examples, a single breath 408 may then be detected for every two detected peaks 414. Thus, a respiratory rate can be calculated based on the number of breathes 412 detected over any period of time. Since only one peak is associated with each sound, false detections of additional breaths may be avoided.

The diagram of FIG. 4C is not intended to indicate that the example detected respiratory rate 400C is to include all of the components shown in FIG. 4C. Rather, the example detected respiratory rate 400C can be implemented using fewer or additional components not illustrated in FIG. 4C (e.g., additional envelopes, peaks, detected breaths, etc.).

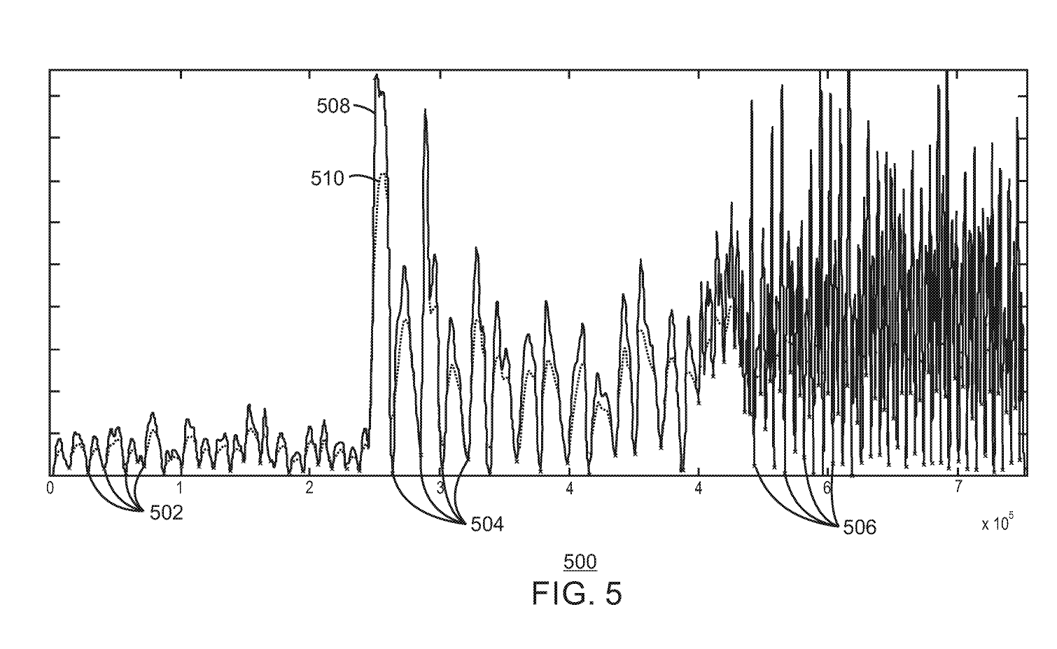

FIG. 5 is a diagram illustrating an example set of detection results. The example set of detection results is generally referred to by the reference number 500 and can be implemented in the computing device 700 below. For example, the set of detection results 500 can be generated using the system 100 of FIG. 1 above, the adaptive respiratory rate detector 728 of the computing device 700 of FIG. 7 below, or the computer readable media 800 of FIG. 8 below.

FIG. 5 shows a set of detection results 500 of an experiment. The results 500 include three types of breaths, including slow breaths 502, normal breaths 504, and rapid shallow breaths 506. In the example of FIG. 5, the accuracy of breath detections in the three types of breaths, including normal breaths 502, deep breathes 504, and shallow breaths 506, are all above 90%. Six detections are missing from the rapid shallow breathing 506, and all the missing detections are at the transition of the sudden change from slow deep breathing 504 to rapid shallow breathing 506. In some examples, breaths can be detected using valley finding rather than peak finding. For example, a received signal can be reversed and the peaks may become valleys. In the example of FIG. 5, the detected valleys in FIG. 5 are indicated using “x”s. In addition, FIG. 5 shows a preprocessed signals 508 as well as adaptive thresholds 510 used for valley finding. For example, the detection results 500 for adaptive bandwidth low-pass filter peak detection are shown in Table 1 below in the bottom row as compared to other static low-pass filters.

TABLE 1
Detection Results for Low-Pass Filters Using Different Bandwidths.

| Band- | | | | | | | | | | |
|---|---|---|---|---|---|---|---|---|---|---|
| width | Normal Breaths (0.65 Hz) | | | Deep Breaths (0.44 Hz) | | | Shallow Breaths (2.2 Hz) | | | Average |
| (Fp) | Missing | Wrong | Accuracy | Missing | Wrong | Accuracy | Missing | Wrong | Accuracy | Accuracy |
| 1.2 | 1 | 0 | 95.00% | 1 | 0 | 92.86% | 70 | 0 | 0.00% | 62.62% |
| 2 | 1 | 0 | 95.00% | 1 | 0 | 92.86% | 69 | 0 | 1.43% | 63.10% |
| 3 | 1 | 1 | 90.00% | 0 | 1 | 92.86% | 34 | 0 | 51.43% | 78.10% |
| 4 | 0 | 4 | 80.00% | 0 | 1 | 92.86% | 23 | 0 | 67.14% | 80.00% |
| 6 | 0 | 9 | 55.00% | 0 | 6 | 57.14% | 0 | 0 | 100.00% | 70.71% |
| 8 | 0 | 15 | 25.00% | 0 | 12 | 14.29% | 0 | 0 | 100.00% | 46.43% |
| Adaptive | 1 | 1 | 90% | 0 | 0 | 100% | 4 | 0 | 94.3% | 94.8% |

In some examples, an accuracy of the detection can be determined using the equation:

Accuracy=((Number of Breaths)−(Number of Wrong Detections)−(Number of Missing Detections))/(Number of Breaths) Eq. 3

For example, according to the Nyquist Theorem, the bandwidth Fp can be at least twice the breathing rate. In the example audio sample used for the detection results above with a sampling rate of 8 k/s, the normal breathing rate is about 0.65 Hz, the deep breathing rate is about 0.44 Hz, and the shallow rapid breathing rate is about 2.2 Hz. As shown in Table 1, the shallow rapid breathing detection is not working when Fp<4, and the normal and deep breathing detection is good when Fp=1.2 or 2. On the other hand, when the bandwidth Fp is too large, more details are showing in the envelope. The envelope may thus be very spiky, with many tiny peaks detectable in one breathing, leading to increased wrong detections. Thus, as shown in Table 1, when the bandwidth Fp>=6, both normal breathing and deep breathing detections are worse.

In some examples, by making the change from slow deep breathing 504 to rapid shallow breathing 506 gradual instead of sudden, there may be fewer missing detections in the detection results 500. For example, the use of a mean breath rate may be used to gradually change a bandwidth of an adaptive low-pass filter. The improved envelope extraction may thus increase detection accuracy. For example, the adaptive bandwidth may gradually follow the change of breathing rate and prevent missed detections accordingly.

The diagram of FIG. 5 is not intended to indicate that the example set of detection results 500 is to include all of the components shown in FIG. 5. Rather, the example set of detection results 500 can be implemented using fewer or additional components not illustrated in FIG. 5 (e.g., additional breathing rates, detections, etc.).

FIG. 6 is a flow chart illustrating a method for detecting respiratory rates in audio signals using an adaptive low-pass filter. The example method is generally referred to by the reference number 600 and can be implemented in the system 100 of FIG. 1 above, the processor 702 of the computing device 700 of FIG. 7 below, or the computer readable media 800 of FIG. 8 below.

At block 602, a processor receives audio including a number of respiratory sounds. For example, the respiratory sounds may be sounds related to inhaling and exhaling of a user.

At block 604, the processor preprocesses the audio to generate preprocessed audio. For example, the processor can preprocess the audio to reduce a DC offset of the audio. In some examples, the processor can take an absolute value of the audio to generate a preprocessed audio.

At block 606, the processor processes the audio using an adaptive low-pass filter with an adaptive bandwidth based on a mean breath rate to generate an envelope. In some examples, the processor can set an initial value for the bandwidth of the adaptive low-pass filter based on an average breath rate. Additionally, in some examples, the processor can set the initial value for the bandwidth of the adaptive low-pass filter based on a detected activity of a user. For example, the detected activity may be running, walking, sitting, etc.

At block 608, the processor detects a plurality of peaks from the envelope. For example, the processor can detect if one or more adaptive thresholds based on peak amplitude and distance between peaks are exceeded.

At block 610, the processor calculates a respiratory rate based on the detected plurality of peaks. For example, the processor can detect a breath based on a number of detected peaks.

At block 612, the processor modifies the bandwidth of the adaptive-low pass filter based on an updated mean breath rate. For example, the processor can increase the bandwidth of the adaptive-low pass filter in response to detecting an increase in the mean breath rate. In some examples, the processor can decrease the bandwidth of the adaptive-low pass filter in response to detecting a decrease in the mean breath rate.

This process flow diagram is not intended to indicate that the blocks of the example process 600 are to be executed in any particular order, or that all of the blocks are to be included in every case. Further, any number of additional blocks not shown may be included within the example process 600, depending on the details of the specific implementation. For example, the processor can also detect a stress level based on the respiratory rate and sending a notification in response to detecting the stress level exceeds a threshold stress level. In some examples, the processor can detect a mood of a user based on the respiratory rate.

Referring now to FIG. 7, a block diagram is shown illustrating an example computing device that can detect respiratory rates in audio signals using an adaptive low-pass filter. The computing device 700 may be, for example, a laptop computer, desktop computer, tablet computer, mobile device, or wearable device, among others. In some examples, the computing device 700 may be embedded into a pair of glasses. The computing device 700 may include a central processing unit (CPU) 702 that is configured to execute stored instructions, as well as a memory device 704 that stores instructions that are executable by the CPU 702. The CPU 702 may be coupled to the memory device 704 by a bus 706. Additionally, the CPU 702 can be a single core processor, a multi-core processor, a computing cluster, or any number of other configurations. Furthermore, the computing device 700 may include more than one CPU 702. In some examples, the CPU 702 may be a system-on-chip (SoC) with a multi-core processor architecture. In some examples, the CPU 702 can be a specialized digital signal processor (DSP) used for image processing. The memory device 704 can include random access memory (RAM), read only memory (ROM), flash memory, or any other suitable memory systems. For example, the memory device 704 may include dynamic random access memory (DRAM).

The memory device 704 can include random access memory (RAM), read only memory (ROM), flash memory, or any other suitable memory systems. For example, the memory device 704 may include dynamic random access memory (DRAM).

The computing device 700 may also include a graphics processing unit (GPU) 708. As shown, the CPU 702 may be coupled through the bus 706 to the GPU 708. The GPU 708 may be configured to perform any number of graphics operations within the computing device 700. For example, the GPU 708 may be configured to render or manipulate graphics images, graphics frames, videos, or the like, to be displayed to a user of the computing device 700.

The memory device 704 can include random access memory (RAM), read only memory (ROM), flash memory, or any other suitable memory systems. For example, the memory device 704 may include dynamic random access memory (DRAM). The memory device 704 may include device drivers 710 that are configured to execute the instructions for detecting respiratory rates. The device drivers 710 may be software, an application program, application code, or the like.

The CPU 702 may also be connected through the bus 706 to an input/output (I/O) device interface 712 configured to connect the computing device 700 to one or more I/O devices 714. The I/O devices 714 may include, for example, a keyboard and a pointing device, wherein the pointing device may include a touchpad or a touchscreen, among others. The I/O devices 714 may be built-in components of the computing device 700, or may be devices that are externally connected to the computing device 700. In some examples, the memory 704 may be communicatively coupled to I/O devices 714 through direct memory access (DMA).

The CPU 702 may also be linked through the bus 706 to a display interface 716 configured to connect the computing device 700 to a display device 718. The display device 718 may include a display screen that is a built-in component of the computing device 700. The display device 718 may also include a computer monitor, television, or projector, among others, that is internal to or externally connected to the computing device 700.

The computing device 700 also includes a storage device 720. The storage device 720 is a physical memory such as a hard drive, an optical drive, a thumbdrive, an array of drives, a solid-state drive, or any combinations thereof. The storage device 720 may also include remote storage drives.

The computing device 700 may also include a network interface controller (NIC) 722. The NIC 722 may be configured to connect the computing device 700 through the bus 706 to a network 724. The network 724 may be a wide area network (WAN), local area network (LAN), or the Internet, among others. In some examples, the device may communicate with other devices through a wireless technology. For example, the device may communicate with other devices via a wireless local area network connection. In some examples, the device may connect and communicate with other devices via Bluetooth® or similar technology.

The computing device 700 further includes a microphone 726. For example, the microphone may include one or more transducers that can convert sound into electrical signals. For example, the microphone 726 may be located in a device such as glasses. As one example, the microphone 726 may be embedded into the nose bridge of a pair of glasses.

The computing device 700 further includes an adaptive respiratory rate detector 728. For example, the adaptive respiratory rate detector 728 can be used to detect respiratory rates in audio signals. The adaptive respiratory rate detector 728 can include an audio receiver 730, an audio preprocessor 732, an adaptive low-pass filter 734, a peak detector 736, a respiratory rate calculator 738, and a filter bandwidth updater 740. In some examples, each of the components 730-740 of the adaptive respiratory rate detector 728 may be a microcontroller, embedded processor, or software module. The audio receiver 730 can receive audio including respiratory sounds. For example, the audio may be an audio signal corresponding to an audio sample of breathing of a user. The audio preprocessor 732 can reduce a DC offset of the audio to generate preprocessed audio, reduce or remove noise, generate an absolute value of audio signals, or any combination thereof. For example, the preprocessed audio can be processed to generate the envelope. In some examples, the audio preprocessor 732 can take an absolute value of the audio to generate a preprocessed audio. The adaptive low-pass filter 734 can process the audio using an adaptive bandwidth based on a detected mean breath rate to generate an envelope. For example, the mean breath rate may be a weighted mean breath rate, with more recent breaths having a higher weight, and older breaths having less weight. The adaptive bandwidth of the adaptive low-pass filter 734 may be adaptive to the mean breath rate. In some examples, an initial bandwidth used by the adaptive low-pass filter is based on an average breath rate. In some examples, an initial bandwidth used by the adaptive low-pass filter is estimated based on a detected activity of a user. For example, the initial bandwidth may be set based on whether the user is running, walking, sitting, etc. In some examples, the adaptive low-pass filter 734 can process preprocessed audio to generate the envelope. The peak detector 736 can detect a plurality of peaks from the envelope. For example, the peak detector 736 can detect peaks using one or more adaptive thresholds based on peak amplitude and distance between peaks. The respiratory rate calculator 738 can calculate a respiratory rate based on the detected plurality of peaks. For example, the respiratory rate calculator 738 can detect a breath rate based on a number of detected peaks. For example, the respiratory rate calculator 738 can detect a breath rate based on a number of detected peaks and the duration of the peaks. The filter bandwidth updater 740 can calculate a mean breath rate based on historical data and update the bandwidth based on the mean breath rate. For example, filter bandwidth updater 740 can calculate a weighted mean breath rate based on historical data. The filter bandwidth updater 740 can then update the bandwidth based on the weighted mean breath rate. For example, a weight of the weighted mean breath rate is based on a regularity of the breathing rate. In some examples, the filter bandwidth updater 740 can increase the bandwidth of the adaptive low-pass filter in response to detecting an increase in the mean breath rate. In some examples, the filter bandwidth updater 740 can decrease the bandwidth of the adaptive low-pass filter in response to detecting a decrease in the mean breath rate.

The block diagram of FIG. 7 is not intended to indicate that the computing device 700 is to include all of the components shown in FIG. 7. Rather, the computing device 700 can include fewer or additional components not illustrated in FIG. 7, such as additional buffers, additional processors, and the like. The computing device 700 may include any number of additional components not shown in FIG. 7, depending on the details of the specific implementation. Furthermore, any of the functionalities of the audio receiver 730, the audio preprocessor 732, the adaptive low-pass filter 734, the peak detector 736, the respiratory rate calculator 738, and the filter bandwidth updater 740, may be partially, or entirely, implemented in hardware and/or in the processor 702. For example, the functionality may be implemented with an application specific integrated circuit, in logic implemented in the processor 702, or in any other device. In addition, any of the functionalities of the CPU 702 may be partially, or entirely, implemented in hardware and/or in a processor. For example, the functionality of the adaptive respiratory rate detector 728 may be implemented with an application specific integrated circuit, in logic implemented in a processor, in logic implemented in a specialized audio processing unit, or in any other device.

FIG. 8 is a block diagram showing computer readable media 800 that store code for detecting respiratory rates in audio signals using an adaptive low-pass filter. The computer readable media 800 may be accessed by a processor 802 over a computer bus 804. Furthermore, the computer readable medium 800 may include code configured to direct the processor 802 to perform the methods described herein. In some embodiments, the computer readable media 800 may be non-transitory computer readable media. In some examples, the computer readable media 800 may be storage media.

The various software components discussed herein may be stored on one or more computer readable media 800, as indicated in FIG. 8. For example, an audio receiver module 806 may be configured to receive audio including respiratory sounds. An audio preprocessor module 808 may be configured to preprocess the audio to reduce a DC offset of the audio. In some examples, the audio preprocessor module 808 may be configured to take an absolute value of the audio to generate a preprocessed audio. An adaptive low-pass filter module 810 may be configured to process the audio using an adaptive low-pass filter with an adaptive bandwidth based on a mean breath rate to generate an envelope. A peak detector module 812 may be configured to detect a plurality of peaks from the envelope. For example, the peak detector module 812 may be configured to detect a peak in response to detecting that one or more adaptive thresholds based on peak amplitude and distance between peaks are exceeded in the audio. A respiratory rate module 814 may be configured to calculate a respiratory rate based on the detected plurality of peaks. For example, the respiratory rate module 814 may be configured to detect a breath based on a number of detected peaks. For example, a breath may be detected for every two detected peaks. A filter bandwidth updater module 816 may be configured to modify the bandwidth of the adaptive-low pass filter based on an updated mean breath rate. For example, the filter bandwidth updater module 816 may be configured to increase the bandwidth of the adaptive-low pass filter in response to detecting an increase in the mean breath rate. The filter bandwidth updater module 816 may be configured to decrease the bandwidth of the adaptive-low pass filter in response to detecting a decrease in the mean breath rate. In some examples, the filter bandwidth updater module 816 may be configured to set an initial value for the bandwidth of the adaptive low-pass filter based on an average breath rate or a detected activity of a user. A mood detector module 818 may be configured to detect a stress level based on the respiratory rate. In some examples, the mood detector module 818 may be configured to send a notification in response to detecting the stress level exceeds a threshold stress level. In some examples, the mood detector module 818 may be configured to detect a mood based on the respiratory rate. For example, the mood may be angry, excited, happy, depressed, etc.

The block diagram of FIG. 8 is not intended to indicate that the computer readable media 800 is to include all of the components shown in FIG. 8. Further, the computer readable media 800 may include any number of additional components not shown in FIG. 8, depending on the details of the specific implementation.

### EXAMPLES

Example 1 is an apparatus for detecting respiratory rates. The apparatus includes an audio receiver to receive audio including respiratory sounds. The apparatus also includes an adaptive low-pass filter to process the audio using an adaptive bandwidth based on a mean breath rate to generate an envelope. The apparatus further includes a peak detector to detect a plurality of peaks from the envelope. The apparatus also further includes a respiratory rate calculator to calculate a respiratory rate based on the detected plurality of peaks.

Example 2 includes the apparatus of example 1, including or excluding optional features. In this example, the apparatus includes a bandwidth updater to calculate the mean breath rate based on historical data and update the bandwidth based on the mean breath rate.

Example 3 includes the apparatus of any one of examples 1 to 2, including or excluding optional features. In this example, the apparatus includes a bandwidth updater to calculate a weighted mean breath rate based on historical data and update the bandwidth based on the weighted mean breath rate, wherein a weight of the weighted mean breath rate is based on a regularity of the breathing rate.

Example 4 includes the apparatus of any one of examples 1 to 3, including or excluding optional features. In this example, the apparatus includes a bandwidth updater to increase the bandwidth of the adaptive low-pass filter in response to detecting an increase in the mean breath rate.

Example 5 includes the apparatus of any one of examples 1 to 4, including or excluding optional features. In this example, the apparatus includes a bandwidth updater to decrease the bandwidth of the adaptive low-pass filter in response to detecting a decrease in the mean breath rate.

Example 6 includes the apparatus of any one of examples 1 to 5, including or excluding optional features. In this example, the apparatus includes an audio preprocessor to reduce a DC offset of the audio to generate preprocessed audio, the preprocessed audio to be processed to generate the envelope.

Example 7 includes the apparatus of any one of examples 1 to 6, including or excluding optional features. In this example, the apparatus includes an audio preprocessor to take an absolute value of the audio to generate a preprocessed audio, the adaptive low-pass filter to process the preprocessed audio to generate the envelope.

Example 8 includes the apparatus of any one of examples 1 to 7, including or excluding optional features. In this example, the peak detector is to detect peaks using one or more adaptive thresholds based on peak amplitude and distance between peaks.

Example 9 includes the apparatus of any one of examples 1 to 8, including or excluding optional features. In this example, the mean breath rate includes a weighted mean breath rate.

Example 10 includes the apparatus of any one of examples 1 to 9, including or excluding optional features. In this example, an initial bandwidth used by the adaptive low-pass filter is estimated based on a detected activity of a user.

Example 11 is a method for detecting respiratory rates. The method includes receiving, via a processor, audio including respiratory sounds. The method also includes processing, via the processor, the audio using an adaptive low-pass filter with and adaptive bandwidth based on a mean breath rate to generate an envelope. The method also further includes detecting, via the processor, a plurality of peaks from the envelope. The method also includes calculating, via the processor, a respiratory rate based on the detected plurality of peaks.

Example 12 includes the method of example 11, including or excluding optional features. In this example, the method includes modifying, via the processor, the bandwidth of the adaptive-low pass filter based on an updated mean breath rate.

Example 13 includes the method of any one of examples 11 to 12, including or excluding optional features. In this example, the method includes increasing the bandwidth of the adaptive-low pass filter in response to detecting an increase in an updated mean breath rate or decreasing the bandwidth of the adaptive-low pass filter in response to detecting a decrease in an updated mean breath rate.

Example 14 includes the method of any one of examples 11 to 13, including or excluding optional features. In this example, detecting the peaks includes detecting that one or more adaptive thresholds based on peak amplitude and distance between peaks are exceeded.

Example 15 includes the method of any one of examples 11 to 14, including or excluding optional features. In this example, calculating the respiratory rate includes detecting a breath based on a number of detected peaks.

Example 16 includes the method of any one of examples 11 to 15, including or excluding optional features. In this example, the method includes preprocessing, via the processor, the audio to reduce a DC offset of the audio.

Example 17 includes the method of any one of examples 11 to 16, including or excluding optional features. In this example, the method includes preprocessing, via the processor, the audio by taking an absolute value of the audio to generate a preprocessed audio.

Example 18 includes the method of any one of examples 11 to 17, including or excluding optional features. In this example, the method includes setting an initial value for the bandwidth of the adaptive low-pass filter based on an average breath rate or a detected activity of a user.

Example 19 includes the method of any one of examples 11 to 18, including or excluding optional features. In this example, the method includes detecting a stress level based on the respiratory rate and sending a notification in response to detecting the stress level exceeds a threshold stress level.

Example 20 includes the method of any one of examples 11 to 19, including or excluding optional features. In this example, the method includes detecting a mood of a user based on the respiratory rate.

Example 21 is at least one computer readable medium for detecting respiratory rates having instructions stored therein that direct the processor to receive audio including respiratory sounds. The computer-readable medium also includes instructions that direct the processor to process the audio using an adaptive low-pass filter with an adaptive bandwidth based on a mean breath rate to generate an envelope. The computer-readable medium further includes instructions that direct the processor to detect a plurality of peaks from the envelope. The computer-readable medium also further includes instructions that direct the processor to calculate a respiratory rate based on the detected plurality of peaks.

Example 22 includes the computer-readable medium of example 21, including or excluding optional features. In this example, the computer-readable medium includes instructions to modify the bandwidth of the adaptive-low pass filter based on an updated mean breath rate.

Example 23 includes the computer-readable medium of any one of examples 21 to 22, including or excluding optional features. In this example, the computer-readable medium includes instructions to increase the bandwidth of the adaptive-low pass filter in response to detecting an increase in the detected breath rate or decrease the bandwidth of the adaptive-low pass filter in response to detecting a decrease in the detected breath rate.

Example 24 includes the computer-readable medium of any one of examples 21 to 23, including or excluding optional features. In this example, the computer-readable medium includes instructions to detect a peak in response to detecting that one or more adaptive thresholds based on peak amplitude and distance between peaks are exceeded in the audio.

Example 25 includes the computer-readable medium of any one of examples 21 to 24, including or excluding optional features. In this example, the computer-readable medium includes instructions to detect a breath based on a number of detected peaks.

Example 26 includes the computer-readable medium of any one of examples 21 to 25, including or excluding optional features. In this example, the computer-readable medium includes instructions to preprocess the audio to reduce a DC offset of the audio.

Example 27 includes the computer-readable medium of any one of examples 21 to 26, including or excluding optional features. In this example, the computer-readable medium includes instructions to preprocess the audio by taking an absolute value of the audio to generate a preprocessed audio.

Example 28 includes the computer-readable medium of any one of examples 21 to 27, including or excluding optional features. In this example, the computer-readable medium includes instructions to set an initial value for the bandwidth of the adaptive low-pass filter based on an average breath rate or a detected activity of a user.

Example 29 includes the computer-readable medium of any one of examples 21 to 28, including or excluding optional features. In this example, the computer-readable medium includes instructions to detect a stress level based on the respiratory rate and send a notification in response to detecting the stress level exceeds a threshold stress level.

Example 30 includes the computer-readable medium of any one of examples 21 to 29, including or excluding optional features. In this example, the computer-readable medium includes instructions to detect a mood of a user based on the respiratory rate.

Example 31 is a system for detecting respiratory rates. The system includes audio receiver to receive audio including respiratory sounds. The system also includes an adaptive low-pass filter to process the audio using an adaptive bandwidth based on a mean breath rate to generate an envelope. The system further includes a peak detector to detect a plurality of peaks from the envelope. The system also further includes a respiratory rate calculator to calculate a respiratory rate based on the detected plurality of peaks.

Example 32 includes the system of example 31, including or excluding optional features. In this example, the system includes a bandwidth updater to calculate the mean breath rate based on historical data and update the bandwidth based on the mean breath rate.

Example 33 includes the system of any one of examples 31 to 32, including or excluding optional features. In this example, the system includes a bandwidth updater to calculate a weighted mean breath rate based on historical data and update the bandwidth based on the weighted mean breath rate, wherein a weight of the weighted mean breath rate is based on a regularity of the breathing rate.

Example 34 includes the system of any one of examples 31 to 33, including or excluding optional features. In this example, the system includes a bandwidth updater to increase the bandwidth of the adaptive low-pass filter in response to detecting an increase in the mean breath rate.

Example 35 includes the system of any one of examples 31 to 34, including or excluding optional features. In this example, the system includes a bandwidth updater to decrease the bandwidth of the adaptive low-pass filter in response to detecting a decrease in the mean breath rate.

Example 36 includes the system of any one of examples 31 to 35, including or excluding optional features. In this example, the system includes an audio preprocessor to reduce a DC offset of the audio to generate preprocessed audio, the preprocessed audio to be processed to generate the envelope.

Example 37 includes the system of any one of examples 31 to 36, including or excluding optional features. In this example, the system includes an audio preprocessor to take an absolute value of the audio to generate a preprocessed audio, the adaptive low-pass filter to process the preprocessed audio to generate the envelope.

Example 38 includes the system of any one of examples 31 to 37, including or excluding optional features. In this example, the peak detector is to detect peaks using one or more adaptive thresholds based on peak amplitude and distance between peaks.

Example 39 includes the system of any one of examples 31 to 38, including or excluding optional features. In this example, the mean breath rate includes a weighted mean breath rate.

Example 40 includes the system of any one of examples 31 to 39, including or excluding optional features. In this example, an initial bandwidth used by the adaptive low-pass filter is estimated based on a detected activity of a user.

Example 41 is a system for detecting respiratory rates. The system includes means for receiving audio including respiratory sounds. The system also includes means for processing the audio using an adaptive bandwidth based on a mean breath rate to generate an envelope. The system further includes means for detecting a plurality of peaks from the envelope. The system also further includes means for calculating a respiratory rate based on the detected plurality of peaks.

Example 42 includes the system of example 41, including or excluding optional features. In this example, the system includes means for calculating the mean breath rate based on historical data and update the bandwidth based on the mean breath rate.

Example 43 includes the system of any one of examples 41 to 42, including or excluding optional features. In this example, the system includes means for calculating a weighted mean breath rate based on historical data and updating the bandwidth based on the weighted mean breath rate, wherein a weight of the weighted mean breath rate is based on a regularity of the breathing rate.

Example 44 includes the system of any one of examples 41 to 43, including or excluding optional features. In this example, the system includes means for increasing the bandwidth of the means for detecting the plurality of peaks in response to detecting an increase in the mean breath rate.

Example 45 includes the system of any one of examples 41 to 44, including or excluding optional features. In this example, the system includes means for decreasing the bandwidth of the means for detecting the plurality of peaks in response to detecting a decrease in the mean breath rate.

Example 46 includes the system of any one of examples 41 to 45, including or excluding optional features. In this example, the system includes means for reducing a DC offset of the audio to generate preprocessed audio, the preprocessed audio to be processed to generate the envelope.

Example 47 includes the system of any one of examples 41 to 46, including or excluding optional features. In this example, the system includes means for taking an absolute value of the audio to generate a preprocessed audio, the means for detecting the plurality of peaks to process the preprocessed audio to generate the envelope.

Example 48 includes the system of any one of examples 41 to 47, including or excluding optional features. In this example, the means for detecting a plurality of peaks is to detect peaks using one or more adaptive thresholds based on peak amplitude and distance between peaks.

Example 49 includes the system of any one of examples 41 to 48, including or excluding optional features. In this example, the mean breath rate includes a weighted mean breath rate.

Example 50 includes the system of any one of examples 41 to 49, including or excluding optional features. In this example, an initial bandwidth used by the means for detecting the plurality of peaks is estimated based on a detected activity of a user.

Not all components, features, structures, characteristics, etc. described and illustrated herein need be included in a particular aspect or aspects. If the specification states a component, feature, structure, or characteristic “may”, “might”, “can” or “could” be included, for example, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

It is to be noted that, although some aspects have been described in reference to particular implementations, other implementations are possible according to some aspects. Additionally, the arrangement and/or order of circuit elements or other features illustrated in the drawings and/or described herein need not be arranged in the particular way illustrated and described. Many other arrangements are possible according to some aspects.

In each system shown in a figure, the elements in some cases may each have a same reference number or a different reference number to suggest that the elements represented could be different and/or similar. However, an element may be flexible enough to have different implementations and work with some or all of the systems shown or described herein. The various elements shown in the figures may be the same or different. Which one is referred to as a first element and which is called a second element is arbitrary.

It is to be understood that specifics in the aforementioned examples may be used anywhere in one or more aspects. For instance, all optional features of the computing device described above may also be implemented with respect to either of the methods or the computer-readable medium described herein. Furthermore, although flow diagrams and/or state diagrams may have been used herein to describe aspects, the techniques are not limited to those diagrams or to corresponding descriptions herein. For example, flow need not move through each illustrated box or state or in exactly the same order as illustrated and described herein.

The present techniques are not restricted to the particular details listed herein. Indeed, those skilled in the art having the benefit of this disclosure will appreciate that many other variations from the foregoing description and drawings may be made within the scope of the present techniques. Accordingly, it is the following claims including any amendments thereto that define the scope of the present techniques.

What is claimed is:

1. An apparatus for detecting respiratory rates, comprising:
an audio receiver to receive audio comprising respiratory sounds;
an adaptive low-pass filter to process the audio using an adaptive bandwidth based on a mean breath rate to generate an envelope;
a peak detector to detect a plurality of peaks from the envelope; and
a respiratory rate calculator to calculate a respiratory rate based on the detected plurality of peaks.

2. The apparatus of claim 1, comprising a bandwidth updater to calculate the mean breath rate based on historical data and update the bandwidth based on the mean breath rate.

3. The apparatus of claim 1, comprising a bandwidth updater to calculate a weighted mean breath rate based on historical data and update the bandwidth based on the weighted mean breath rate, wherein a weight of the weighted mean breath rate is based on a regularity of the breathing rate.

4. The apparatus of claim 1, comprising a bandwidth updater to increase the bandwidth of the adaptive low-pass filter in response to detecting an increase in the mean breath rate.

5. The apparatus of claim 1, comprising a bandwidth updater to decrease the bandwidth of the adaptive low-pass filter in response to detecting a decrease in the mean breath rate.

6. The apparatus of claim 1, comprising an audio preprocessor to reduce a DC offset of the audio to generate preprocessed audio, the preprocessed audio to be processed to generate the envelope.

7. The apparatus of claim 1, comprising an audio preprocessor to take an absolute value of the audio to generate a preprocessed audio, the adaptive low-pass filter to process the preprocessed audio to generate the envelope.

8. The apparatus of claim 1, wherein the peak detector is to detect peaks using one or more adaptive thresholds based on peak amplitude and distance between peaks.

9. The apparatus of claim 1, wherein the mean breath rate comprises a weighted mean breath rate.

10. The apparatus of claim 1, wherein an initial bandwidth used by the adaptive low-pass filter is estimated based on a detected activity of a user.

11. A method for detecting respiratory rates, comprising:
receiving, via a processor, audio comprising respiratory sounds;
processing, via the processor, the audio using an adaptive low-pass filter with and adaptive bandwidth based on a mean breath rate to generate an envelope;
detecting, via the processor, a plurality of peaks from the envelope; and
calculating, via the processor, a respiratory rate based on the detected plurality of peaks.

12. The method of claim 11, comprising modifying, via the processor, the bandwidth of the adaptive-low pass filter based on an updated mean breath rate.

13. The method of claim 12, comprising increasing the bandwidth of the adaptive-low pass filter in response to detecting an increase in the updated mean breath rate or decreasing the bandwidth of the adaptive-low pass filter in response to detecting a decrease in the updated mean breath rate.

14. The method of claim 11, wherein detecting the peaks comprises detecting that one or more adaptive thresholds based on peak amplitude and distance between peaks are exceeded.

15. The method of claim 11, wherein calculating the respiratory rate comprises detecting a breath based on a number of detected peaks.

16. The method of claim 11, comprising preprocessing, via the processor, the audio to reduce a DC offset of the audio.

17. The method of claim 11, comprising preprocessing, via the processor, the audio by taking an absolute value of the audio to generate a preprocessed audio.

18. The method of claim 11, comprising setting an initial value for the bandwidth of the adaptive low-pass filter based on an average breath rate or a detected activity of a user.

19. The method of claim 11, comprising detecting a stress level based on the respiratory rate and sending a notification in response to detecting the stress level exceeds a threshold stress level.

20. The method of claim 11, comprising detecting a mood of a user based on the respiratory rate.

21. At least one computer readable medium for detecting respiratory rates having instructions stored therein that, in response to being executed on a computing device, cause the computing device to:
receive audio comprising respiratory sounds;
process the audio using a low-pass filter with an adaptive bandwidth based on a mean breath rate to generate an envelope;
detect a plurality of peaks from the envelope; and
calculate a respiratory rate based on the detected plurality of peaks.

22. The at least one computer readable medium of claim 21, comprising instructions to modify the bandwidth of the adaptive-low pass filter based on an updated mean breath rate.

23. The at least one computer readable medium of claim 22, comprising instructions to increase the bandwidth of the adaptive-low pass filter in response to detecting an increase in the updated mean breath rate or decrease the bandwidth of the adaptive-low pass filter in response to detecting a decrease in the updated mean breath rate.

24. The at least one computer readable medium of claim 23, comprising instructions to detect a peak in response to detecting that one or more adaptive thresholds based on peak amplitude and distance between peaks are exceeded in the audio.

25. The at least one computer readable medium of claim 24, comprising instructions to detect a breath based on a number of detected peaks.
